(19) United States
(12) Patent Application Publication
(10) Pub. No.: US 2017/0252196 A1
(43) Pub. Date: Sep. 7, 2017

(54) DEVICE FOR ALLEVIATING THE COMPLAINTS OF A TRAPEZIOMETACARPAL JOINT DISEASE FROM THE DEGENERATIVE, INFLAMMATORY, AND RHEUMATIC SPECTRUM

(71) Applicant: Christine MEYER, Meppen (DE)

(72) Inventor: Christine MEYER, Meppen (DE)

(21) Appl. No.: 15/328,402

(22) PCT Filed: Jun. 9, 2015
(86) PCT No.: PCT/DE2015/100229

§ 371 Date: May 19, 2017

(30) Foreign Application Priority Data
Jul. 22, 2014 (DE) 20 2014 103 362.9

(51) Int. Cl. A61F 5/01

(52) U.S. Cl. CPC A61F 5/0118

(57) ABSTRACT

A device for alleviating the complaints of a trapeziometacarpal joint disease from the degenerative, inflammatory, and rheumatic spectrum. The load on the trapeziometacarpal joint changed by arthrosis can be dynamically relived by means of said device. The device includes a splint, which is detachably fastened to the wrist on one side and reaches under the first thumb joint of the diseased hand on the other side. The splint accommodates the thumb in a natural resting position of thumb. If the thumb is bent, then, below the thumb joint, the thumb stresses the splint against the retainer fastened to the wrist as a result of the radial motion or bending of the thumb and thereby lifts the trapeziometacarpal joint minimally from a usual seat thereof. A gap thus arises between the joint bodies that engage with each other. The joint surfaces no longer painfully rub against each other.

Carpometacarpal osteoarthritis in particular involves a disease of the trapeziometacarpal joint, which can result in non-specific signs of attrition, associated with a reduction in the sliding layer of the cartilaginous joint, or even complete loss thereof, together with increasingly painful inflammation and sensitivity to pressure. Moreover, at a secondary level, the capsular ligament apparatus is damaged and weakened.

Previously known treatment methods immobilize the joint, as a result of which movements and loads that are felt to cause pain are suppressed to greater or lesser effect.

DE 3006 362 A1 describes an invention comprising two open supports secured on the forearm, in which invention, among other things, the thumb is inserted into a sleeve extending to the end of the splint, such that the whole hand, including the wrist, is unable to move.

DE 3519493 A1 discloses a device for immobilizing the trapeziometacarpal joint, in which device the thumb and the hand are fixed by being at least partially enclosed by a solid shaped body. On account of the anatomical configuration of the splint, the hand remains in its natural position and thus provides an immobilization of the trapeziometacarpal joint and also of the metacarpo-phalangeal joint of the thumb.

WO 2011/110420 A1 moreover claims a device which has the purpose of providing a specific and complete immobilization of the trapeziometacarpal joint, thereby reliably suppressing the painful movement of the joints that are affected by the arthritis.

All of the inventions have the disadvantage that, as a result of the joint being immobilized, the capsular ligament and muscle apparatus slackens within a short space of time, leading to progressively limited mobility together with worsening of the pain symptoms.

Moreover, the desired immobilization can be achieved only if the sometimes plastically deformable and thus anatomically adapted splint firmly encloses the parts of the body that are to be locked. The blood flow is impaired as a result of this, and pressure points lead to sensitivity and chafing.

The immobilization has the effect that even very slight physical activity is almost entirely suppressed, and the pinch grip that can be formed by thumb and middle finger is made much more difficult or indeed impossible.

DE 20 2012 009 952 U1 departs from the aforementioned principles by not only immobilizing the trapezio-metacarpal joint but also expanding it by means of a wing-shaped padding which is adhesively affixed in the anatomically adapted orthosis and which is made of a resiliently cushioned material. Pressure acting on the tip of the thumb, as happens in particular in a pinch grip, is thus transferred via the anatomically adapted orthosis to the metacarpus, which as it were increases the pressure on the padding and increasingly expands the joint shells rubbing on each other.

This invention has the disadvantage that the trapezio-metacarpal joint rests firmly enclosed in a splint. The pressure which is exerted on the splint, and which is further increased by the adhesively affixed padding, has the effect that stresses can occur at points, or even pressure sores, when the device is being worn. Moreover, the adaptation of the orthosis entails not inconsiderable difficulties, since the padding presses the joint apart only if exactly aligned, otherwise it even increases the pain caused by the underlying disease.

The object of the invention is to make available a device by means of which the load on the arthritic trapeziometacarpal joint is dynamically relieved, this being associated with the advantage that the capsular ligament and muscle apparatus is supported in its natural movement by permanent tensioning and untensioning and it therefore does not slacken.

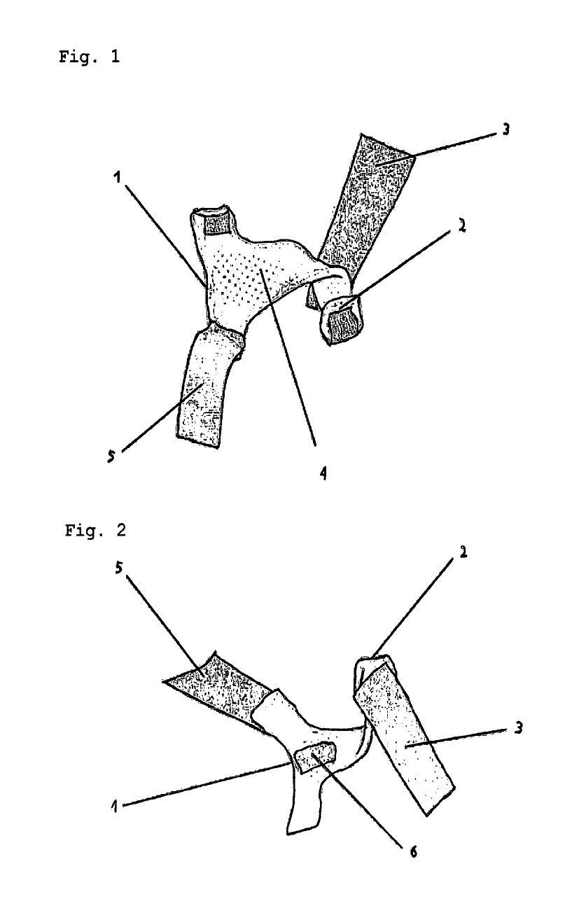

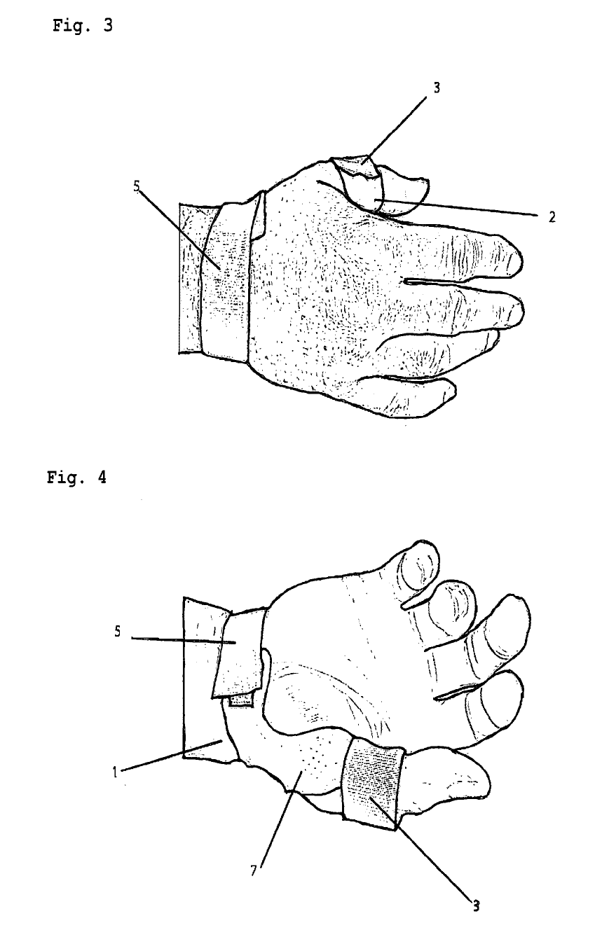

FIGS. 1 and 2 show the device, while FIGS. 3 and 4 show the device fitted in place.

The invention comprises a splint (FIGS. 1-4) which can be releasably fixed (5) at one end on the wrist of the patient and at the other end (3) below the first joint of the thumb of the diseased hand. It consists of a reinforced thermoplastic which partially encloses (1) the wrist, lies with an exact fit on the thenar eminence (7) and terminates (2) with a corkscrew-shaped left winding below the first joint of the thumb.

In its basic position, the splint is designed such that it receives the thumb in its natural position of rest.

If the thumb is now guided to the middle finger for the pinch grip, the thumb (2), fixed below the first joint of the thumb, tensions the splint (4), on account of the radial movement thereof, against the retainer (1) secured on the wrist and thereby lifts the trapeziometacarpal joint of the thumb minimally from its usual seat, as a result of which a gap arises between the mutually engaging joint heads, such that the latter, on account of the movement, rub against each other to a lesser extent, or indeed not at all, which brings about a noticeable reduction in pain.

The effect when bending the first phalanx of the thumb is comparable. As a result of the splint being designed as an abutment against the wrist, the enclosed joint of the thumb levers the trapeziometacarpal joint minimally from the joint seat and thus relieves the pressure on the opposing bones of the joint.

The dynamic process of adapted unloading means that the rest of the capsular ligament and muscle apparatus is constantly stressed and supported. The further advantage of the invention is that, because of the small amount of material used, the device feels pleasant to wear, in particular on account of the small coverage of the skin, and does not cause any discomfort.

In a preferred embodiment, a padding is worked into the splint below the trapeziometacarpal joint (6).

In another preferred embodiment, the splint is made of perforated plastic.

1-3. (canceled)

4. A thumb orthosis, comprising:
a rail configured to be detachably connected to a wrist and a thumb of a wearer, the rail comprising:
a wrist portion configured to extend along the wrist at a position proximal of a carpometacarpal joint of the thumb;
a thumb portion extending from the wrist portion and configured to extend laterally over a ball of a hand of the wearer and along the thumb, the thumb portion having a corkscrew shape that rotates toward a palm of the hand, the thumb portion terminating proximal of an interphalangeal joint of the thumb;
a wrist strap configured to releasably connect the wrist portion to the wrist;
a thumb strap configured to releasably connect the thumb portion to the thumb at a position proximal of the interphalangeal joint of the thumb.

5. The thumb orthosis of claim 4, wherein the rail comprises plastic.

6. The thumb orthosis of claim 4, wherein the rail comprises perforated plastic.

7. The thumb orthosis of claim 4, further comprising a pad positioned on or in the rail.

8. The thumb orthosis of claim 7, wherein the pad is positioned below the carpometacarpal joint when the orthosis is attached to the wearer.

9. The thumb orthosis of claim 4, wherein the wrist and thumb straps include hook-and-loop fasteners.

10. The thumb orthosis of claim 4, wherein a free end of each strap connects to a fixed portion of the respective strap.

11. The thumb orthosis of claim 4, wherein the wrist portion of the rail extend along portions of a front side and a rear side of the wrist.

12. The thumb orthosis of claim 4, wherein the thumb portion is configured to connect to the thumb along a phalanx bone of the thumb.

13. The thumb orthosis of claim 4, wherein the rail has a rigid construction.

14. A method of supporting a thumb with a thumb orthosis, the method comprising:
providing a thumb orthosis having a rail, the rail having a wrist portion and a thumb portion, the thumb portion extending from the wrist portion;
positioning the wrist portion along a wrist of a wearer at a position proximal of a carpometacarpal joint of a thumb of the wearer;
positioning the thumb portion extending laterally over a ball of the hand and along the thumb, the thumb portion having a corkscrew shape that rotates toward a palm of the hand, the thumb portion terminating proximal of an interphalangeal joint of the thumb;
connecting the wrist portion to the wrist, and connecting the thumb portion to the thumb at a location proximal of the interphalangeal joint of the thumb.

15. The method of claim 14, wherein the thumb orthosis further comprises a wrist strap connected to the wrist portion, and connecting the wrist portion to the wrist includes wrapping a free end of the wrist strap around the wrist.

16. The method of claim 14, wherein the thumb orthosis further comprises a thumb strap connected to the thumb portion, and connecting the thumb portion to the thumb includes wrapping a free end of the thumb strap around the thumb.
